(19) United States
(12) Patent Application Publication
(10) Pub. No.: US 2010/0093852 A1
(43) Pub. Date: Apr. 15, 2010

(54) Pharmaceutical composition comprising shikonin derivatives from lithospermum erythrorhizon for treating or preventing diabetes mellitus and the use thereof

(76) Inventors: Ji Ho Park, Seoul (KR); Sun Yeou Kim, Gyeonggi-do (KR); Tong Ho Kang, Seoul (KR); Eun Ju Hwang, Gyeonggi-do (KR); Chul Hoon Kang, Gyeonggi-do (KR)

Correspondence Address: Olson & Cepuritis, LTD., 20 NORTH WACKER DRIVE, 36TH FLOOR, CHICAGO, IL, 60606, US

(21) Appl. No.: 12/312,943

(22) PCT Filed: Dec. 12, 2006
(86) PCT No.: PCT/KR2006/005414

§ 371 Date: Jun. 1, 2009

(51) Int. Cl. A61K 31/216; C07C 69/007; A61P 3/10

(52) U.S. Cl. 514/510; 560/129; 560/221

(57) ABSTRACT

The present invention relates to a composition comprising shikonin compounds, especially, isobutyryl shikonin, β-β-dimethylacryl shikonin, isovaleryl shikonin, and α-methyl-n-butyryl shikonin as an active ingredient for the prevention and treatment of diabetes mellitus and the use thereof. The composition of the present invention showed stimulating effect on the release of insulin in the beta cell of the pancreas due to inhibitory effect on K_{ATP} ion channel of beta cell in pancreas together with promoting effect on the increase of calcium concentration. Therefore, it can be used as the therapeutics, health functional food or food additive for treating and preventing diabetes mellitus.

### TECHNICAL FIELD

The present invention relates to a pharmaceutical composition comprising shikonin derivative compounds isolated from Lithospermum erythrorhizon for the prevention and treatment of diabetes mellitus and the use thereof.

### BACKGROUND ART

Diabetes can be classified into two categories: one is an insulin-dependent diabetes mellitus (Type I) occupying about 10% patients among the patients suffering from diabetes and occurring in younger children aged less than 20 years old, which is called as infant diabetes caused by genetic factor; another is a insulin-independent diabetes mellitus (Type II) mainly occurring in older people aged more than 40 years old, which is caused by reduced susceptibility to insulin in peripheral organ such as muscle, or adipose tissue and the like resulting from various factors, for example, hypokinesia, obesity, hyperphagia, stress and so on. About 50%-80% patients suffering with type II diabetes could be treated by diet therapy and exercise therapy and the disease is not required to insulin treatment and not dependent to insulin.

Recently, the occurrence rate of type II diabetes has been sharply increased in proportion with the number of fatty people has been increased. It has been reported that the insulin secreted from beta cell in pancreas releases to supplement as much as the necessary amount of reduced insulin in normal adult however the release of insulin is also reduced in accordance with the reduced response of beta cell in patient suffering with type II diabetes (Kahn, B. B., Cell 92, pp593-596, 1998; Kahn, B. B., Nature Genet. 20, pp223-225, 1998).

There has been also reported that type II diabetes directly correlated with ATP sensitive potassium ion channel (K_{ATP} ion channel) (Tarasov. A. et al., Diabetes 53 (3), ppS113-S122, 2004). The K_{ATP} ion channel of beta cell in pancreas is opened to let inner K⁺ ion out-put when the blood glucose concentration is lowered, which causes to membrane hyperpolarization. In the end, the Ca²⁺ channel is closed resulting in blocking insulin release. On the contrary, outer glucose is influxed into beta cell to increase cellular ATP and to let K_{ATP} ion channel be closed when the blood glucose concentration is increased, which causes to membrane de-polarization. In the end, outer Ca²⁺ ion is influxed into inner cell through Ca²⁺ ion channel resulting in promoting insulin release. However, the above-described mechanism does not work well in diabetes patient.

Glibenclamide, a representative sulfonylurea type-diabetic treating agent which causes the K_{ATP} ion channel of beta cell in pancreas to be closed compulsorily has been used in the market as a diabetes treating agent till now.

At present, various blood glucose lowering drugs have been conventionally used to treat diabetes however they show various adverse response such as hypoglycemia, hepato-toxicity, weight-gain, lactic acidosis etc.

Therefore, there have been needed to develop more safe and potent drugs to treat diabetes till now and focused on the development from natural products having safe properties and various effective ingredients recently.

Lithospermum erythrorhizon Siebold & Zuccarini belonged to Boraginaceae is distributed over mountain region in Korea, Japan, China etc and the root of plant comprises acetylshikonin, isobutyryl shikonin, β,β-dimethylacryl shikonin, isovaleryl shikonin, α-methyl-n-butyryl shikonin and β-hydroxyisovaleryl shikonin etc.

There have been reported that shikonin compounds isolated from Lithospermum erythrorhizon show various activities: for example, anticancer activity (Xin, C. et al., Phytotherapy Research, 16 (3). pp. 199-209, 2002); anti-oxidative activity (Weng, X C. et al., Food Chemistry, 69 (2), pp. 143-146, 2000) and so on.

However, there has been not reported or disclosed on the diabetic activity of shikonin compound isolated from the root of Lithospermum erythrorhizon in any of above cited literatures, the disclosures of which are incorporated herein by reference.

Therefore, the present inventors have endeavored to find the diabetic activity of shikonin compounds through various experiments such as inhibition effect on K_{ATP} ion channel of beta cell in pancreas, promoting effect on the increase of proinsulin mRNA expression, glucose intolerance test using by male ICR mouse etc and finally confirmed that the shikonin compounds isolated from the root of Lithospermum erythrorhizon such as isobutyryl shikonin, β,β-dimethylacryl shikonin, isovaleryl shikonin, α-methyl-n-butyryl shikonin and β-hydroxyisovaleryl shikonin show potent anti-diabetic activity.

### DISCLOSURE OF INVENTION

### Technical Problem

According to one aspect, the present invention provides to a pharmaceutical composition comprising shikonin derivative compounds isolated from Lithospermum erythrorhizon for the prevention and treatment of diabetes mellitus.

The present invention also provides a use of above compounds for the preparation of pharmaceutical composition to treat and prevent diabetes mellitus in mammal or human.

The present invention also provides a health food or food additives comprising above compounds for the prevention or alleviation of diabetes mellitus.

### Technical Solution

Accordingly, it is an object of the present invention to provide a pharmaceutical composition comprising shikonin compounds represented by following general chemical formula (I) as an active ingredient, a pharmaceutically acceptable carrier, additive or excipient, for the prevention and treatment of diabetes mellitus:

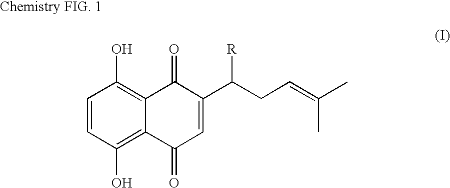

Wherein,

R is OCOCH(CH₃)₂, OCOCH═C(CH₃)₂, OCOCH₂CH(CH₃)₂ or OCOCH(CH₃)CH₂CH₃.

Preferably, the shikonin compounds represented by general chemical formulae I are selected from isobutyryl shikonin, β,β-dimethylacryl shikonin, isovaleryl shikonin, α-methyl-n-butyryl shikonin or β-hydroxyisovaleryl shikonin.

It is another object of the present invention to provide a use of the shikonin compounds represented by general chemical formulae I for the preparation of therapeutic agent for the treatment and prevention of diabetes mellitus in human or mammal.

It is an object of the present invention to provide a method of treating or preventing diabetes mellitus comprising administering to said mammal an effective amount of the shikonin compounds represented by general chemical formulae I, together with a pharmaceutically acceptable carrier thereof.

The shikonin compounds of the present invention may be prepared from the extract of Lithospermum erythrorhizon in accordance with the following preferred embodiment:

For example, Lithospermum erythrorhizon is cut, crushed, dried in lyophilizer and left at less than 0° C., preferably, −20° C., in refrigerator. The dried powder is mixed with 1 to 20-fold, preferably, about 3 to 10-fold volume of distilled water, lower alcohols having 1 to 4 carbon atoms such as methanol, ethanol, butanol and the like, or the mixtures thereof, preferably, the mixture solvent of water and ethanol in a mixed ratio ranging from 60 to 100%, preferably, 85% ethanol based on the weight of the powder, and then is subjected to ultra-sonication extraction for the period ranging from 5 minutes to 5 hours, preferably, 1 hours, and repeated 2 to 5 times, consecutively. The residue is filtered to obtain the supernatant to be concentrated with rotary evaporator, and then concentrated under reduced pressure to obtain crude extract of Lithospermum erythrorhizon.

The crude extract of Lithospermum erythrorhizon prepared in aforementioned step is concentrated and the residue is suspended in distilled water. The suspension is further fractionated into two fractions, i.e., water fraction and non-polar organic solvent fraction such as mixed fraction (chloroform: ethanol fraction) using by chloroform: ethanol (2:1) mixture solution and the non-polar organic solvent fraction is subjected to HPLC to isolate purposed shikonin compounds such as isobutyryl shikonin, β,β-dimethylacryl shikonin, isovaleryl shikonin, α-methyl-n-butyryl shikonin or β-hydroxyisovaleryl shikonin.

The inventive compounds represented by general formula (I) can be transformed into their pharmaceutically acceptable salt and solvates by the conventional method well known in the art. For the salts, acid-addition salt thereof formed by a pharmaceutically acceptable free acid thereof is useful and can be prepared by the conventional method. For example, after dissolving the compound in the excess amount of acid solution, the salts are precipitated by the water-miscible organic solvent such as methanol, ethanol, acetone or acetonitrile to prepare acid addition salt thereof and further the mixture of equivalent amount of compound and diluted acid with water or alcohol such as glycol monomethylether, can be heated and subsequently dried by evaporation or filtrated under reduced pressure to obtain dried salt form thereof.

As a free acid of above-described method, organic acid or inorganic acid can be used. For example, organic acid such as methansulfonic acid, p-toluensulfonic acid, acetic acid, trifluoroacetic acid, citric acid, maleic acid, succinic acid, oxalic acid, benzoic acid, lactic acid, glycolic acid, gluconic acid, galacturonic acid, glutamic acid, glutaric acid, glucuronic acid, aspartic acid, ascorbic acid, carbonylic acid, vanillic acid, hydroiodic acid and the like, and inorganic acid such as hydrochloric acid, phosphoric acid, sulfuric acid, nitric acid, tartaric acid and the like can be used herein.

Further, the pharmaceutically acceptable metal salt form of inventive compounds may be prepared by using base. The alkali metal or alkali-earth metal salt thereof can be prepared by the conventional method, for example, after dissolving the compound in the excess amount of alkali metal hydroxide or alkali-earth metal hydroxide solution, the insoluble salts are filtered and remaining filtrate is subjected to evaporation and drying to obtain the metal salt thereof. As a metal salt of the present invention, sodium, potassium or calcium salt are pharmaceutically suitable and the corresponding silver salt can be prepared by reacting alkali metal salt or alkali-earth metal salt with suitable silver salt such as silver nitrate.

The pharmaceutically acceptable salt of the present compound comprise all the acidic or basic salt which may be present at the compounds, if it does not indicated specifically herein. For example, the pharmaceutically acceptable salt of the present invention comprise the salt of hydroxyl group such as the sodium, calcium and potassium salt thereof; the salt of amino group such as the hydrogen bromide salt, sulfuric acid salt, hydrogen sulfuric acid salt, phosphate salt, hydrogen phosphate salt, dihydrophosphate salt, acetate salt, succinate salt, citrate salt, tartarate salt, lactate salt, mandelate salt, methanesulfonate(mesylate) salt and p-toluenesulfonate (tosylate) salt etc, which can be prepared by the conventional method well known in the art.

Accordingly, the present invention also provides a pharmaceutical composition comprising shikonin compounds represented by following general chemical formula (I) prepared by above-described procedure as an active ingredient for the treatment and prevention of diabetes mellitus.

The inventive composition for treating and preventing diabetes may comprise above described compound as 0.01˜99.9% by weight based on the total weight of the composition, of which ratio is not intended to limit thereto and can be varied according to the condition of patient, the type of disease and the progress of disease.

The pharmaceutical formulation may be prepared by using the composition in accordance with any of the conventional procedures. In preparing the formulation, the active ingredient is preferably admixed or diluted with a carrier or enclosed within a carrier, which may be in the form of a capsule, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

The pharmaceutical formulations can be administered via various routes including oral, transdermal, subcutaneous, intravenous and intramuscular introduction. A typical daily dose of the active ingredient may range from about 0.01 mg/kg to 10 g/kg body weight, preferably 1 mg/kg to 1000 mg/kg body weight, and can be administered in a single dose or in divided dose. However, it should be understood that the amount of the active ingredient actually administered ought to be determined in light of various relevant factors including the condition to be treated, the chosen route of administration, the age, sex and body weight of the individual patient, and the severity of the patient's symptom; and. Therefore, the above dose should not be intended to further illustrate the present invention without limiting its scope.

The pharmaceutical composition of the present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Also, the present invention provide a health food composition comprising shikonin compounds represented by following general chemical formula (I) for the prevention and improvement of diabetes mellitus by adding other food components besides the compounds of the present invention.

The health food disclosed herein comprise various food such as confectionary, sweetening food, ice cream, modified milk food, meat product, fish food, bean-curd product, jelly product, edible oil, noodle, tea product, beverage, special nutrient food, health care food, seasoning food, ice, ginseng product, gimchi food, jerked meat and the like.

Above health food can be contained in health food, health beverage etc, and may be prepared by the form comprising conventional food, a powder, granule, tablet, chewing tablet, capsule, beverage etc.

To develop for health food, examples of addable food comprising above extracts of the present invention are various food, beverage, gum, vitamin complex, health improving food and the like, and can be used as power, granule, tablet, chewing tablet, capsule or beverage etc.

Also, the present invention provide a food additive comprising shikonin compounds represented by following general chemical formula (I) for the prevention and improvement of diabetes by adding to conventional food.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, α-tocopherol, rosemary extract, vitamin C, green tea extract, licorice root extract, chitosan, tannic acid, phytic acid etc.

Above described composition therein can be added to food, additive or beverage, wherein, the amount of above described compound in food or beverage may generally range from about 0.1 to 80 w/w %, preferably 1 to 50 w/w % of total weight of food for the health food composition and 1 to 30 g, preferably 3 to 10 g on the ratio of 1000 of the health beverage composition.

Providing that the health beverage composition of present invention contains above described extract as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100□ of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

The above described food additive of the present invention may be 20 to 90% high concentrated liquid, power, or granule type.

Similarly, the above described food additive of the present invention can comprise additionally one or more than one of lactose, casein, dextrose, glucose, sucrose and sorbitol.

Inventive compound of the present invention have no toxicity and adverse effect therefore; they can be used with safe.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally ranging from about 0 to 20 w/w % per 100 w/w % present composition.

Above described food includes various food, for example, dried fruit, dried vegetable, fruit juice, vegetable juice, mixed juice, chip, noodle, confectionary, modified milk food, processed meat product, modified fish food, fermented milk food, bean-curd product, grain food, bakery, seasoning, beverage, licorice, herb and the like.

Also, the present invention provide a health functional food comprising shikonin compounds represented by following general chemical formula (I) as an active ingredient for the prevention and improvement of diabetes.

Examples of addable food comprising aforementioned compound therein are various food, beverage, gum, vitamin complex, health improving food and the like.

Above described the composition therein can be added to food, additive or beverage for the prevention and alleviation of diabetes.

Prevention and the treatment of diabetes and diabetic complications, wherein, the amount of above described compound in food or beverage may generally range from about 0.01 to 15 w/w %, preferably 1 to 10 w/w % of total weight of food for the health food composition and 0.02 to 10 g, preferably 0.3 to 1 g on the ratio of 100□ of the health beverage composition.

Providing that the health beverage composition of the present invention contains above described compound as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100□ of present beverage composition.

The present invention is more specifically explained by the following examples.

However, it should be understood that the present invention is not limited to these examples in any manner.

### Advantageous Effects

Accordingly, the shikonin compounds of the present invention showed stimulating effect on the release of insulin in the beta cell of the pancreas due to inhibitory effect on K_{ATP} ion channel of beta cell in pancreas together with promoting effect on the increase of calcium concentration. Therefore, it can be used as the therapeutics, health functional food or food additive for treating and preventing diabetes mellitus.

### BRIEF DESCRIPTION OF THE DRAWINGS

The above and other objects, features and other advantages of the present invention will more clearly understood from the following detailed description taken in conjunction with the accompanying drawings, in which;

FIG. 1 shows the scheme of extraction and fractionation to isolate shikonin compounds from the extract of Lithospermum erythrorhizon;

FIG. 2 shows the result of HPLC analysis of the extract of Lithospermum erythrorhizon;

FIG. 3 presents the effect of various concentrations of the extract of Lithospermum erythrorhizon on K_{ATP} ion channel;

FIG. 4 presents the comparison between the effect of the extract of Lithospermum erythrorhizon and GBC as a positive control on K_{ATP} ion channel;

FIG. 5 represents the effect of the extract of Lithospermum erythrorhizon and shikonin compounds on K_{ATP} ion channel;

FIG. 6 represents the effect of the extract of Lithospermum erythrorhizon on the increase of mRNA expression in proinsulin;

FIG. 7 depicts the inhibitory effect of the extract of Lithospermum erythrorhizon on the glucose absorption;

FIG. 8 depicts an effect of the extract of Lithospermum erythrorhizon on insulin-resistant type II diabetes.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### Mode for the Invention

### EXAMPLE 1

### Preparation of the Crude Extract of Lithospermum erythrorhizon

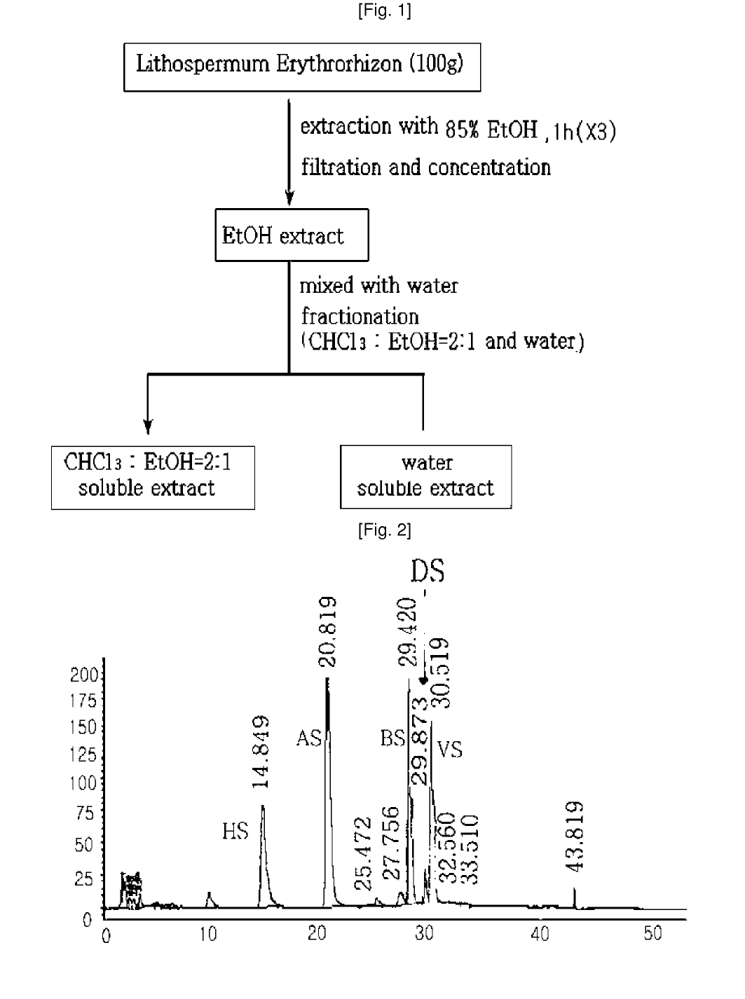

1 kg of Lithospermum erythrorhizon purchased from Kyung-dong market located in Seoul were washed, cut into the with of 1 cm, dried in freeze dryer and kept at −20° C. in refrigerator. 100 g of the powder was subjected to extraction with ultrasonicatior using by 11 of 85% ethanol for 1 hr at 50° C. and the extraction was repeated by 5 times to collect crude extract. The extract was filtered and the filtrate was concentrated to afford 350 g of crude extract of Lithospermum erythrorhizon. (designated as “LE.S” hereinafter, See FIG. 1)

### EXAMPLE 2

### Purification and Isolation of the Shikonin Compounds

350 g of the crude extract prepared in Example 1 was suspended in distilled water and fractionated into water fraction and non-polar solvent soluble fraction (designated as “CHCl₃:Ethanol (2:1) fraction” hereinafter) using by distilled water and mixed organic solvent (CHCl₃:Ethanol=2:1). The CHCl₃:Ethanol (2:1) fraction was subjected to HPLC by using a solvent mixture of 60% acetonitrile and 40% distilled water of which ratio of acetonitrile was gradually increased to 100% after 15-30 mins. The solvent system was maintained till 30-40 mins after the elution and the ratio of acetonitrile was reduced to 60% after 40 mins.

Using by the condition of semi-preparative HPLC, the shikonin compounds represented by following chemical formula 1 to 4 were isolated:

### 2-1. Isobutyryl Shikonin (Nacalai Tesque INC., Kyoto, Japan) (1)

Molecular Weight: 358.39

Molecular Formula: C₂₀H₂₂O₆

Purity: ≧98% (HPLC)

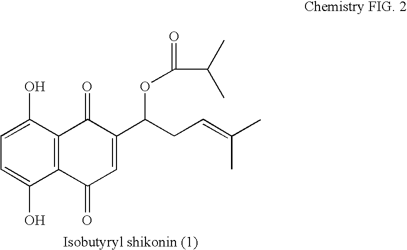

2-2. β,β-dimethylacryl Shikonin (Nacalai Tesque INC., Kyoto, Japan) (2)

Molecular Weight: 370.40

Molecular Formula: C₂₁H₂₂O₆

Purity: 98% (HPLC)

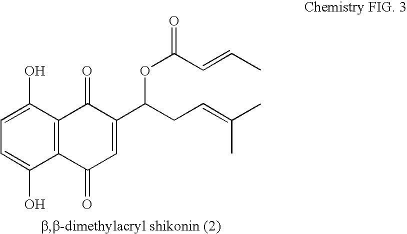

### 2-3. Isovalerylshikonin (Nacalai Tesque INC., Kyoto, Japan) (3)

Molecular Weight: 372.41

Molecular Formula: C₂₁H₂₄O₆

Purity: ≧98% (HPLC)

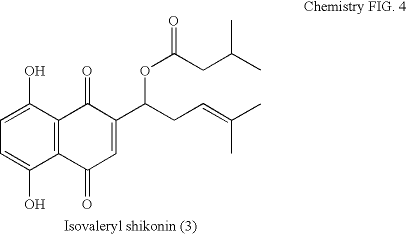

2-4. α-methyl-n-butyryl Shikonin (4) (Nacalai Tesque INC., Kyoto. Japan) (4)

Molecular Weight: 372.41

Molecular Formula: C₂₁H₂₄O₆

Purity: ≧98% (HPLC)

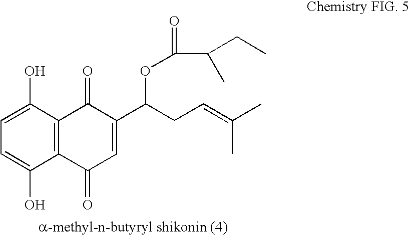

### REFERENCE EXAMPLE 1

### HIT-T15 Cell Culture

The HIT-T15 cells was incubated on RPMI 1640 culture medium containing 10% (v/v) horse serum, 2.5% fetal bovine serum (v/v) and 1% penicillin-streptomycin in 5% CO₂ incubator at 37° C.

### EXPERIMENTAL EXAMPLE 1

### Electro-Physiological Experiment

Based on the theory that Type II diabetes is directly correlated with K_{ATP} ion channel in beta cell of the pancreas (Tarasov. A. et al., Diabetes, 53 (3), pp. S113-5122, 2004), present inventors investigated the effect of the extract of Lithospermum erythrorhizon and the shikonin compounds isolated therefrom in Examples on K_{ATP} ion channel in beta cell of the pancreas as follows:

1-1. Procedure

Ionic current was recorded by using patch clamp amplifier (EPC-9, Heka Elektronik, Lambrecht, Germany) with typical whole cell patch clamp method.

A tourmaline glass tube (borosilicate glass capillary; Sutter instrument Co., USA) was pulled out by using puller (DMZ-Universal puller; Dagan Co., USA) as a measuring electrode of which resistance was maintained at 6-9M Ω when solution was filled in inner electrode.

The cell attached cover glass was placed onto the microscope and the extra-cellular fluid was let to flow at the speed of 0.1˜0.5 ml/min with gravity.

The solution filled in electrode at the determination of K_{ATP} electric current consists of 10 mM NaCl, 102 mM KCl, 1 mM CaCl₂, 1 mM MgCl₂, 10 mM HEPES, 0.1 mM Na₂-ATP, 1 mM Na₂-GTP and 10 mM EGTA (pH 7.2).

The capacitance and series resistance of cell membrane was amended by 80% for recording voltage clamp and low-pass filter was set to 1 kHz during the test. The cell solution was substituted with 110 mM barium solution containing 110 mM BaCl₂, 10 mM HEPES and 10 mM Glucose to measure K_{ATP} electric currency.

The test result was analyzed by Pulse/Pulsefit (v8.65) software (Heka Elektronik, Lambrecht, Germany) and all the experiment was performed at room temperature.

1-2. Effect of Various Concentrations of Extract on K_{ATP} Ion Channel

Various concentrations of the extract of Lithospermum erythrorhizon were tested to determine the effect on K_{ATP} ion channel. At the result, the electric currency of K_{ATP} ion channel was reduced at 20 mV in a dose dependent manner.

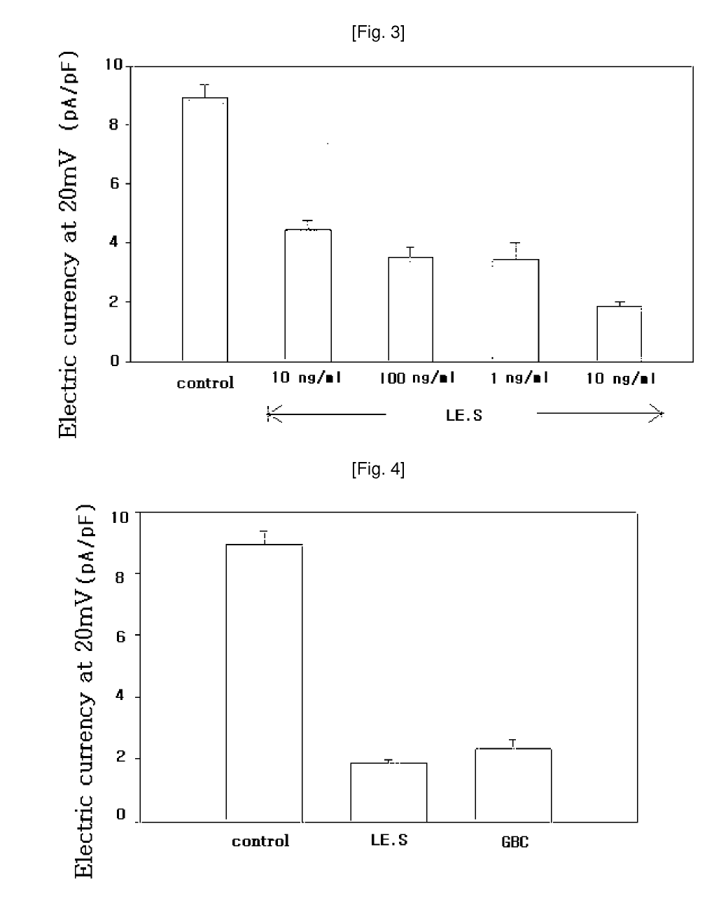

10 ng/ml, 100 ng/ml and 10□/ml treatment groups of LE.S showed decreased electric currency by about 50%, 66% and 75% respectively (See FIG. 3).

1-3. Comparison Between the Effect of LE.S and Positive Control Group on K_{ATP} Ion Channel

To compare the effect of LE.S and positive control group on K_{ATP} ion channel. GBC (Glienclamide), well-known K_{ATP} ion channel blocker was used as a positive control. At the result, the treatment group with LE.S also reduced the electric currency of K_{ATP} ion channel in a similar level to positive control. Accordingly, it has been confirmed that the extract of Lithospermum erythrorhizon (LE.S) showed potent blocking activity of K_{ATP} ion channel (See FIG. 4).

1-4. Comparison Between the Effect of LE.S and Shikonin Compounds on K_{ATP} Ion Channel

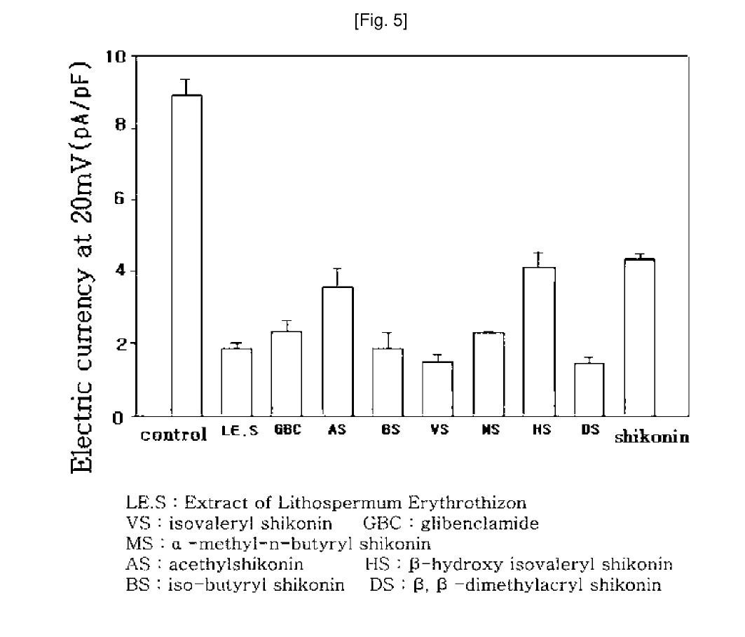

To compare the effect of LE.S and shikonin compounds isolated from LE.S on K_{ATP} ion channel. The effect of various concentrations of shikonin compounds on K_{ATP} ion channel was also determined. At the result, the treatment group with shikonin compounds such as iso-butyryl shikonin (BS), β,β-dimethylacryl shikonin (DS), isovaleryl shikonin (VS), α-methyl-n-butyryl shikonin (MS) or β-hydroxyisovaleryl shikonin, especially, VS and DS showed potent inhibiting effect on the electric currency of K_{ATP} ion channel (See FIG. 5).

### EXPERIMENTAL EXAMPLE 2

### Effect on the Increase of Proinsulin mRNA Expression

To verify whether LE.S increases the release of insulin where the glucose level was increased in pancreatic cell or not, the effect on the proinsulin mRNA expression was determined.

At the result, the test groups treated with 0.1□/ml and 1□/ml of LE.S increase the proinsulin mRNA expression by 13% and 20% respectively comparing with the comparative group administrated group compare with comparative test, each 13%, about 20% was increased (See FIG. 5).

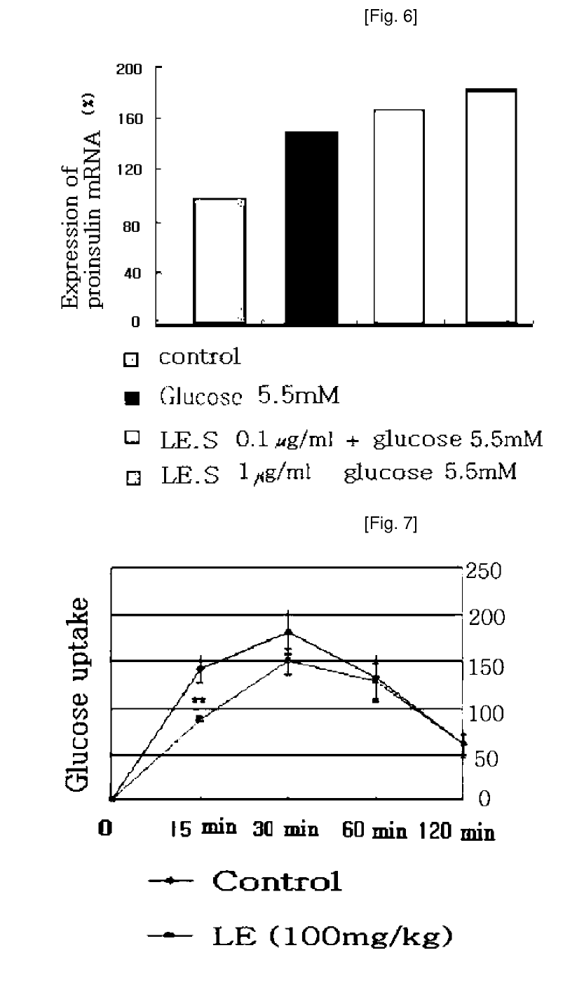

Pancreas is main organ to secrete insulin and the increase of proinsulin mRNA expression increase insulin release. Accordingly, it has been confirmed that the extract of Lithospermum erythrorhizon (LE.S) lowered the blood glucose resulting from the increased insulin release where the blood glucose was increased (See FIG. 6).

### EXPERIMENTAL EXAMPLE 3

### Glucose Tolerance Test

7 weeks old male ICR mouse procured from Chung-ang Experiments Co. Ltd located in Korea was used in the experiment. The mice have been starved for 18 hours and 2.0 g/kg of sucrose was orally administrated into the mice. Physiological saline solution was orally administrated into mice with sucrose simultaneously as a negative control group. LE.S was orally administrated into mice with sucrose simultaneously in an amount of 100mg/kg as a test group and the blood was collected at routine times, i.e., 1, 15, 30, 60 and 120 mins from tail vein to determine the glucose level by one touch ultra glucose test kit (Life Scan, Inc. U.S.A.). The test results were expressed as means SE and the significance verification was performed by way of student t-test (**p<0.01, n=5).

At the result, the sucrose tolerance was reached to maximum blood glucose level at 30 mins, which showed normal glucose kinetic profile. 15, 30, 60 and 120 minutes after the sucrose tolerance, the blood glucose level of LE.S treatment group was more reduced by 39%, 18%, 4% and 1% respectively, comparing with the level of normal negative group, especially, the glucose uptake was sharply inhibited at 30 and 60 mins.

Accordingly, it has been confirmed that the extract of Lithospermum erythrorhizon (LE.S) can be used as a potent special diet for diabetic patients or anti-diabetic agent due to their potent inhibiting activity of initial glucose uptake at food intake (See FIG. 7).

### EXPERIMENTAL EXAMPLE 4

### Effect of the Extract of Lithospermum erythrorhizon (LE.S) on Type II Diabetes

LE.S was orally administrated into db/db mice daily and the blood was collected at routine times, i.e., 0, 1, 2, 3 and 4 days from tail vein to determine the glucose level by one touch ultra glucose test kit (Life Scan, Inc. U.S.A.).

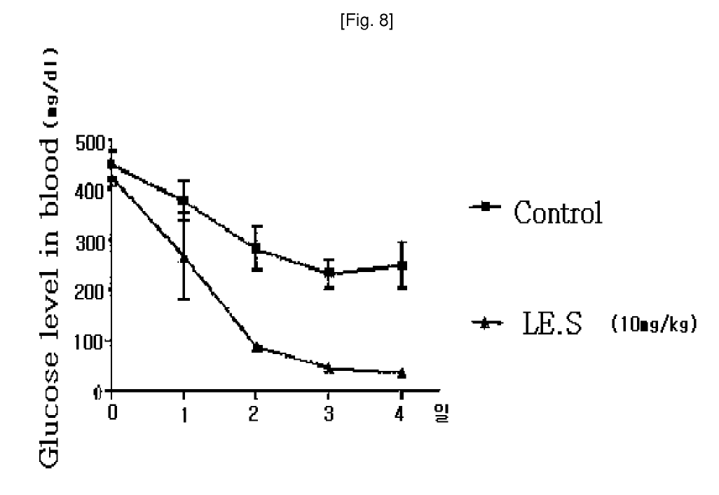

At the result, the glucose level of LE.S treatment group was more reduced than that of negative control group, especially, at 3 and 4 day (See FIG. 8).

Hereinafter, the formulating methods and kinds of excipients will be described, but the present invention is not limited to them. The representative preparation examples were described as follows.

Preparation of Powder

α-methyl-butyryl shikonin 100 mg

Corn Starch 100 mg

Lactose 100 mg

Talc 10 mg

Powder preparation was prepared by mixing above components and filling sealed package.

Preparation of Tablet

Isobutyryl shikonin 100 mg

Corn Starch 100 mg

Lactose 100 mg

Magnesium stearate 2 mg

Tablet preparation was prepared by mixing above components and entabletting.

Preparation of Capsule

β,β-dimethylacryl shikonin 100 mg

Lactose 50 mg

Magnesium stearate 1 mg

Tablet preparation was prepared by mixing above components and filling gelatin capsule by conventional gelatin preparation method.

Preparation of Liquid

α-methyl-n-butyryl shikonin 100 mg

Sugar 10 g

Polysaccharide 10 g

Lemon flavor optimum amount

Distilled water optimum amount

Liquid preparation was prepared by dissolving active component, and then filling all the components in 100□ ample and sterilizing by conventional liquid preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described above, the composition of the present invention showed potent anti-diabetic activity and safety therefore it can be used as the therapeutics, health functional food or food additive for treating and preventing diabetes mellitus.

While the invention has been described with respect to the above specific embodiments, it should be recognized that various modifications and changes may be made to the invention by those skilled in the art which also fall within the scope of the invention as defined by the appended claims.

1. A pharmaceutical composition comprising shikonin compounds represented by following general chemical formula (I) as an active ingredient, a pharmaceutically acceptable carrier, additive or excipient, for the prevention and treatment of diabetes mellitus: 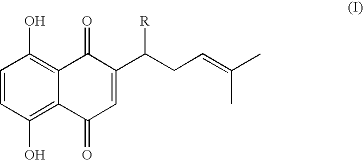
Wherein,
R is OCOCH(CH₃)₂, OCOCH═C(CH₃)₂, OCOCH₂CH(CH₃)₂ or OCOCH(CH₃)CH₂CH₃.

2. The pharmaceutical composition according to claim 1 wherein the shikonin compounds are selected from isobutyryl shikonin, β,β-dimethylacryl shikonin, Isovaleryl shikonin or α-methyl-n-butyryl shikonin.

3. A use of a shikonin compounds represented by general chemical formula (I) as set forth in claim 1, for the preparation of therapeutic agent for the treatment and prevention of diabetes mellitus in human or mammal.

4. A method of treating or preventing diabetes mellitus and diabetic complications, wherein the method comprises administering a therapeutically effective amount of the shikonin compound represented by general chemical formula (I) as set forth in claim 1.

5. A food additive comprising shikonin compounds represented by general chemical formula (I) as set forth in claim 1, for the prevention and improvement of diabetes mellitus.

6. The food additive according to claim 5, wherein said health food is provided as powder, granule, tablet, capsule or beverage type.

7. The food additive according to claim 5, wherein the shikonin compounds are selected from isobutyryl shikonin, β,β-dimethylacryl shikonin, Isovaleryl shikonin or α-methyl-n-butyryl shikonin.

8. A health functional food comprising shikonin compounds represented by general chemical formula (I) as set forth in claim 1 as an active ingredient for the prevention and improvement of diabetes mellitus.

9. The health functional food according to claim 8, wherein the shikonin compounds are selected from Isobutyryl shikonin, β,β-dimethylacryl shikonin, Isovaleryl shikonin or α-methyl-n-butyryl shikonin.
